(19) United States
(12) Patent Application Publication
(10) Pub. No.: US 2003/0009290 A1
(43) Pub. Date: Jan. 9, 2003

(54) Predictive method

(76) Inventors: Bradford E. Billet, New York, NY (US); Sreekanth Thumrugoti, Hyderabad (IN)

Correspondence Address: GIBSON, DUNN & CRUTCHER LLP, Suite 4100, 1801 California Street, Denver, CO, 80202-2641, US

(21) Appl. No.: 09/846,734

(22) Filed: Apr. 30, 2001

(51) Int. Cl. G01N 33/48; G06F 19/00

(52) U.S. Cl. 702/19

(57) ABSTRACT

A method and system for forecasting using pattern recognition and extension software. Models of the present invention select patterns from a library that match historical data and extend them into the future to make forecasts that can be used with a variety of predictive technologies.

### FIELD OF THE INVENTION

The present invention relates to the field of methods for predicting the occurrence of identifiable events, using numerical modeling based on past occurrences. The method is best implemented using computer aided numerical processing. The invention has wide applicability in predicting important events in science, medicine, meteorology, sociology, disease control, manufacturing and other areas. More specifically, but without limitation, the system can be used in forecasting vector-borne and other kinds of serious or fatal disease, as well as the demand for beneficial and life-saving drugs; forecasting agricultural pests and agricultural diseases for use in the chemical and pesticide manufacturing industries; assisting the pharmaceutical industries in the design, testing, synthesis and manufacture of new therapeutic molecules and compounds; increasing the speed of microprocessors; optimizing power grid operations by forecasting demand and equipment failures, and minimizing transmission and distribution losses; forecasting customer behavior for so-called Customer Relationship Management; forecasting the failure of critical equipment to allow for timely service and repair; forecasting the behavior of customers for e-commerce sites; and forecasting interest rates for banks and other financial institutions.

### BACKGROUND OF THE INVENTION

It is generally recognized that events are produced by causes. In the simplest model, event “B” is directly produced by the operation of cause “A” over some necessary short or long period of time. In more complex models, event “B” is produced by several causes. These several causes may produce event “B” by their simple additive effect, or only if occurring in a particular sequence, or only if occurring at precise relative times, or only upon some combination of the foregoing. Further, a causative event may in fact be the absence of a certain event. In other words, a causative event can be the absence at an appropriate time of a blocking event.

There have been efforts to express causative forces and effects numerically. Indeed, much of mathematics is based on the premise that effects can be expressed as functions of their causes. Thus, the simple formula f(x)=Kx expresses the concept that a given outcome is a function of variable “x” and constant “K.” More particularly, it is equal to “K” multiplied times “x.” More complex equations can be utilized to express an outcome as a more complicated function of a cause or as a function of additional causative factors, including a time variable.

One approach to predictive modeling is to gain a thorough understanding of the causative mechanism. In economic theory, for example, there is some understanding of the mechanism by which high interest rates curtail economic expansion. With a complete understanding of that mechanism, one can numerically model it, at least in theory. The difficulty is that in practice many other variables come into play in highly complicated ways. In areas such as predicting weather, the outbreak of disease, economic performance and other real-world occurrences, the causative factors are extremely complicated and intertwined. Some of these causative factors are unfathomable, or at least hopelessly complex, such as human emotion and psychology.

Another approach which is related to the method of the present invention focuses on empirical models in which the model is fitted to the data with less regard for the scientific underpinnings of the causative mechanism. In some ways these empirical numerical models are less appealing than numeric models derived from an understanding of the causative mechanisms. Empirical numeric models may seem “unscientific” by tying events to causative variables without an understanding of the causation mechanism. Moreover, they are largely ineffective in predicting events that have never occurred in the past, because for such events there is no database from which to construct the empirical model (although this might be addressed in part by using extrapolation or projection techniques). In addition, the numeric model that is empirically derived may erroneously fail to consider certain important causative factors simply because these factors were not present at the past occurrences upon which the model is based, or were present but are not recognized in the empirical modeling as being a causative factor. Empiric numeric modeling is very useful despite these limitations.

The current forecasting tools depend on extracting knowledge from large databases and interpreting this knowledge to forecast future events. This process of extracting knowledge is sometimes called data mining. There are two principal approaches to this process: verification/user-driven data mining, and data driven data mining.

Traditionally the goal of identifying and utilizing information hidden in data has proceeded via query generators and data interpretation systems. In verification driven data mining, a user formats a theory about a possible relation in a database and converts this hypothesis into a query. For example, a user might hypothesize about the relationship between industrial sales of color copiers and customers' specific industries. He or she would generate a query against a data warehouse and segment the results into a report. Typically, the generated information provides a good overview.

There are several limitations to verification driven data mining. First, it is based on a hunch. In the above example, the hunch is that a company's industry correlates with the number of copiers it buys or leases. Second, the quality of the extracted information depends on the user's interpretation of the results, and is thus subject to error. Multi-factor analyses identify the relationships among factors that influence the outcome of copier sales. Pearson product-moment correlation measures the strength and direction of the relationship between each database field and the dependent variable. One of the problems with this approach, aside from its resource intensity, is that the techniques tend to focus on tasks in which all the attributes have continuous or ordinal values. Many of the attributes are also parametric. The following are among the methodologies followed:

A linear classifier, for instance, assumes that a relationship is expressible as a linear combination of the attribute values.

Statistical methodology assumes normally distributed data—an often tenuous assumption in the real world of corporate data warehouses.

Manual (top-down approach) data mining stems from the need to know facts, such as regional sales reports stratified by type of business.

Automatic (bottom-up) data mining comes from the need to discover the factors that influence these sales.

Even some sophisticated AI-based tools that use case-based reasoning, a nearest neighbor indexing system, fuzzy (continuous) logic, and genetic algorithms don't qualify as data mining tools since their queries also originate with the user. Certainly the way these tools optimize their search on a data set is unique, but they do not perform autonomous data discovery. Neural networks, polynomial networks, and symbolic classifiers do qualify as true automatic data mining tools because they autonomously interrogate the data for patterns. Neural networks, however, often require extensive care and feeding—they can only work with preprocessed numeric, normalized, scaled data. They also need a fair amount of tuning such as the setting of a stopping criterion, learning rates, hidden nodes, momentum coefficients, and weights. And their results are not always comprehensible. In data driven data mining, symbolic classifiers are examples. These use machine learning technology, and hold great potential as data mining tools for corporate data warehouses. These tools do no require any manual intervention in order to perform their analysis. Their strength is their ability to automatically identify key relationships in a database—to discover rather than confirm trends or patterns in data and to present solutions in usable business formats. They can also handle the type of real-world business data that statistical and neural systems have to “scrub” and scale.

Most of these symbolic classifiers are also known as rule-induction programs or decision-tree generators. They use statistical algorithms or machine-learning algorithms such as ID3, C4.5, AC2, CART, CHAIRd, CN2, or modifications of these algorithms. Symbolic classifiers split a database into classes that differ as much as possible in their relation to a selected output. That is, the tool partitions a database according to the results of statistical tests conducted on an output by the algorithm instead of by the user.

Machine learning algorithms use the data—not the user's hypotheses—to automate the stratification process. To start the process, the type of data mining tool requires a “dependent variable” or outcome, such as copier sales, which should be a field in the database. The rest is automatic. The tool's algorithm tests a multitude of hypotheses in an effort to discover the factors or combination of factors (e.g., business type, location, number of employees) that have the most influence on the outcome. The algorithm engages in a kind of “20 Questions” game. Presented with a database of 5,000 buyers and 5,000 non-buyers of copiers, the algorithm asks a series of questions about the values of each record. Its goal is to classify each sample into either a buyer or non-buyer group. The tool processes every field in every record in the database until it sufficiently splits the buyers from the non-buyers and learns the main differences between them. Once the tool had learned the crucial attributes, it can rank them in order of importance. A user can then exclude attributes that have little or no effect on targeting potential new customers. Most data mining tools generate their findings in the format of “if then” rules. Symbolic Classifiers do have some advantages. For example:

Symbolic classifiers do not require an intensive data preparation effort. This is a convenience to end-users who freely mix numeric, categorical, and date variables.

They provide broad analyses. Unlike traditional statistical methods of data analysis which require the user to stratify a database into small subgroups in order to maximize classification or prediction, data mining tools use all the data as the source of their analysis.

These tools formulate their solutions in English. They can extract “if-then” business rules directly from the data based on tests that they conduct for statistical significance. They can optimize business conditions by providing answers to decision-makers on important questions. Almost all of the current symbolic classifier-type data mining tools incorporate a methodology for explaining their findings. They also tabulate model error-rates for estimating the accuracy of their predictions. In a business environment where small changes in strategy translate to millions of dollars, this type of insight can quickly equate to profits. Some of these tools can also generate graphic decision trees, which display a summary of significant patterns and relationships in the data.

Symbolic classifiers also have some critical disadvantages:

Many of today's analytic tools have capabilities for performing sophisticated user-driven queries. They are, however, limited in their abilities to discover hidden trends and patterns in a database.

All these trends and patterns can reflect only the past. They try to visualize future from past data.

These trends and patterns tend to change. If the same example of color copiers, sales of a new model, if it is superior to existing models, follow a pattern. Initially sales start to pick up slowly as the customers require time to see the advantages and get used to them. Suddenly sales rise exponentially on the customer acceptance. They reach a plateau; then, because of emergence of some new technology/new model, they start falling. It becomes a very steep fall soon. Then they disappear altogether. The database containing the past data does not reflect this pattern. This is the reason that their ability to forecast future events is limited. Most of the time their accuracy levels hover around 50%-60% levels.

Even apart from its inherent limitations, prior art empiric numeric modeling lacks any systematic methodology for establishing the necessary numeric sequences. The result is that the numeric sequences that ultimately are chosen may not be the best ones available for correlating the chosen variables with real-life occurrences. A better method is desired for establishing numeric sequences predictive of real-world events based on historic data. The present invention includes such a method.

### SUMMARY OF THE INVENTION

The present invention is a new paradigm in forecasting technologies. It is data driven, pattern recognizing and extension software. All the current forecasting models try to interpret historical data by the way of establishing relationships and extracting hidden knowledge, and base their predictions on these interpretations. The mathematical model of the present invention selects one of the patterns from its library that matches with the historical data and extends it into the future to make the forecast. This results in several important advantages. There are no assumptions on relationships. The input data need not be distributed. The user need not originate queries, but, can instead perform autonomous data discovery. It completely automates the data analysis for extracting hidden knowledge and does not require any human intervention. It discovers trends and presents solutions in usable business formats. It can handle real world business data directly without any need to scrub the data.

The pattern library component of the present invention is very large. It uses both horizontal and vertical pattern recognizing methods. The horizontal patterns identify inter-relationships between various parameters (such as price of an item and customer decision to purchase it) and the knowledge that can be extracted from, and their relationship to, the eventual event. The vertical patterns project these parametric values into the future.

Both the vertical and horizontal patterns are called numeric sequences. Using these Number Sequences (NSS), the present invention builds an N-Dimensional Numeric Space (NDNS), taking time on x-axis. The NDNS can be extended into future, so behaviors of each one of the parameters as well as the actual event can be extended into the future.

In a preferred embodiment, the system utilizes a numeric “space” constructed of “n” dimensions, wherein “n” is typically much more than three. The x-axis represents time in suitable increments, another axis represents a number indicative of the event being predicted, and other axes represent parameters that correlate with the event. The number of parameter axes is equal to the number of parameters correlated with the event.

In the case of a single parameter correlated with the event, the “space” is ordinary three-dimensional space wherein one axis represents time, a second axis represents a numeric scale indicative of the occurrence of the event, and the third axis represents the parameter that correlates with or is a function of the first two. The variables are thus plotted on multi-dimensional x-y axes with an integrated paradigm for said axes. A three dimensional plot of this space is easy to visualize, in which a “strand” or other geometric figure shows the interrelationship among these three variables. This “strand” which is also called NSS is similar to the double helix of DNA. It consists of two strings of numbers. One string of numbers represents historical data of the event and a selected parameter. The second string represents the corresponding patterns selected from the pattern library. A relationship between these two strings of the strand will be established. Once this strand or other geometric figure is established based on historic data, it can be mathematically characterized or “modeled.” It can then be projected or extrapolated into the time region beyond the historic data, i.e., the future. This is possible as the pattern string is of infinite length. Using the already established relationship between the pattern string and historical data, the string representing future data will be drawn. The same concept applies when using more than one parameter correlating with time and the predicted event, although of course the concept is then impossible to visualize since it involves a space” of greater than three dimensions.

The method in a preferred embodiment utilizes software written in the Java brand programming language. Such software is platform independent and can be used on most machines. Six modules may be used: data reader, diary of events, iterative generator, forecaster, communicator, and optimizer.

The data reader facilitates the input of data from one or more databases such as ORACLE, SYSBASE, INGRES brand databases or others. The report is made to a FOXPRO brand or flat data file. The data reader may also utilize web-based software to allow access to data from remote servers over a network such as the Internet.

The diary of events module establishes a relationship between factors that are causative or otherwise correlated with the predicted event by reading data from the data reader module and employing a pattern recognition tool.

The interactive generator works in tandem with the diary of events module to generate an n-dimensional numeric space (sometimes referred to as “NDNS”) and a set of corresponding numeric sequence strands (sometimes referred to as “NSS”) using a set of interrelated formulae. The NDNS and NSS are generated using an iterative process that repeatedly compares the calculated results against the actual historic data.

The forecaster module utilizes the iterative generator to produce predictions of future events. Such predictions can be short-term or long-term or both. As additional events that are the subject of the predictive system occur, the historic database can be updated to tune the system for better future predictions.

The communication module is used to transmit or otherwise communicate predictions to appropriate persons. For example, a system used for predicting disease outbreaks transmits predictions to appropriate health authorities, a system used for predicting flooding transmits predictions to appropriate rescue or aid groups, or can communicate a warning prior to a system failure in the case of power grids or machinery or other mechanized devices.

The optimizer module of the method assists the users in improving upon the forecasted results. The optimizer contains a built in simulator. This simulator provides user with an opportunity to perform “what if” analysis. Here the user can change values of various parameters (theoretically) and see how these changes effect the forecasted results. This module also provides user an opportunity to fix time and intensity of an event based on which this method calculates and recommends feasible ranges of various parametric values. Once the user takes necessary steps to keep the parametric values within the range recommended by the method, occurrence of the forecasted event can be arrested, deferred or intensified as per requirements.

Once this has occurred, one can begin using the above process to forecasts values for each of the parameters. Knowing the values or the weights of each of the parameters is key in the optimization process. These weights may change based on the interrelationship of the other inputs.

For example, consider the factors relating to purchase of ice-cream. Although many factors are involved in the purchase ice cream, not all have the same weight, and not all have the same weight throughout the elapsed time. The weather, temperature, price, taste, and location, among other additional factors, will influence the purchase. In the summer, the temperature has a great weight, over 90 degrees; that factor outweighs all others. Conversely, in the winter, when the temperature is 32 degrees, taste may outweigh all of the other factors. Each weight is calculated in the NSS and NS over an elapsed time, making a multidimensional x-y axis with an integrated paradigm.

Once the set of algorithms are known, the program can query the desired result and work backwards to notify the user what parameter values must change in order to increase or decrease the projected outcome based upon the ND, NS and NSS integrated paradigm utilizing synchronization and discrepancies.

The invention constructs both the numeric sequence strands as well as the numeric sequence values in an integrated multidimensional paradigm. Once these keys are known, for an event, the invention uses the mathematical formulas in reverse to get the optimum result by recommending the change in the input values, such as price or delivery times to increase sales in the case of ice cream.

As explained in greater detail below, the methodology of the present invention has broad application in predicting the occurrence of events for which past data is available. Applications include, for example, the forecasting of vector-borne diseases, so that preventive measures can be taken and to allow predictions of the demand for treatments such as pharmaceuticals. Similarly, the method can be used to forecast the incidence of agricultural blights or pests and the corresponding demand for pesticides or other chemical treatments. In the pharmaceutical industry, the method assists in designing new drugs in the form of particular molecules or compounds by predicting their efficiency, and also in implementing their manufacture. The method is also useable in designing microprocessors optimized for speed, efficiency, low cost or ease of manufacture. In the area of utility service, the system accurately predicts customer demand in order to optimize power grid operations. In all areas, the system can be used to predict equipment failures, so that appropriate equipment maintenance and replacement can be undertaken on a timely basis. In retailing and wholesaling, the system can be used in Customer Relationship Management and the forecasting of customer behavior at e-commerce sites or other sale sites. The system can even be used by banks and other financial institutions to predict interest rates.

The system can also be used in numeric processing. Traditionally, computers perform numeric processing by emphasizing classic arithmetic calculations. This can be very processor-intensive. The present invention allows a processor instead to recognize patterns in numeric processing and to substitute these patterns for the step of arithmetic computation.

### DETAILED DESCRIPTION OF THE INVENTION

In a most basic embodiment, the invention involves identifying a set of formulae (or numeric sequence strands), and then establishing a very high number of patterns utilizing combinations of those formulae. These patterns are created independently of time variable or data. These patterns are applied to data sets. Then, the patterns are repeatedly compared to the calculated values until an acceptable relationship can be discerned. That relationship can then be extended into the future to predict the future occurrence of the event in question. A detailed description follows.

This process can be visualized as encompassing a set of numeric sequences which produce numeric sequence “strands.” As a first step of the invention in a preferred embodiment, a set of Numeric Sequences is developed. The Numeric Sequences are functions of elapsed time. Each can therefore be plotted in two dimensions, such as with the Numeric Sequence on the y axis and elapsed time on the x axis. Additionally, it is also plotted on multi-dimensional x-y axes with an integrated paradigm for said axes. Many formulae can be used for these Numeric Sequences which are functions of elapsed time, but it has been found that formulae that correspond to patterns in nature are those effective in the invention.

Numeric Sequence Strands (NSS) are built using multi-dimensional x-y axes with an integrated paradigm for said axes. The following are the three phases of the process, which are described in greater detail below.

1. Building NSS for the event data.

2. Building NSS for each one of the parameters.

3. Discrepancy.

Building NSS for the Event Data.

This phase is divided into the following six steps.

1. Collecting historical data of the event.

2. Calculating Numeric Sequence (NS) values.

3. Building multi-dimensional x-y axes with an integrated paradigm.

4. Constructing NSS.

5. Synchronization.

6. Extending NSS into future, i.e., beyond historical data for forecasting.

Collection of Historical Data on the Event.

Historical data on the event intensity is collected at the available frequency. The effectiveness of the forecast increases with larger data sets. The forecast also becomes more accurate as the historical period for which data is collected increases.

Calculating Numeric Sequence (NS) Values.

NS values are calculated by using a set of NS formulae. In these calculations initial Elapsed Time (ET) value will be zero. In each iteration ET is incremented by finest possible interval between collected historical data. This is called Time Interval (TI). The precision of the forecast depends on this TI duration. The finer this TI duration, the more precise the forecast. It is important to note that except for this time interval, this method does not use historical data for the calculation of NS.

Building Multi-Dimensional x-y Axes with an Integrated Paradigm.

Elapsed Time (ET) is plotted on x-axis. Here TI is the unit of measurement. NS values are plotted on y-axes. As there is more than one NS value, there will be more than one y-axes. This can be viewed as a number of two-dimensional planes superimposed on one another. For the given ET there will be a set of NS values NS1, NS2 . . . . , NS36. This set of NS values is called a pattern. This means there will be one new pattern each time when ET increased by TI. As ET can be extended infinitely, there can be a very large set of patterns. One important fact point about these patterns is that they are repeated only after a very large number of units. These patterns when extended on the time scale, i.e., on the x-axis, and visualized (as there is more than one y-axis it cannot be drawn physically), they resemble a crumpled ribbon. This is called a numeric string (because each one of the points in this string is a number). The space thus created is called multi-dimensional x-y axes. The process that followed to build this space is called integrated paradigm.

Constructing NSS.

The NSS is drawn upon the multi-dimensional x-y axes space. It resembles a double helix of DNA, as it consists of two strings of numbers. The first string is made of NS patterns. The second string is made with values from historical data. ET for the first occurrence of the events is taken as zero. The rest of the historical data is plotted as per the elapsed time between events.

Synchronization.

Synchronization establishes an arithmetic relationship between these two strings. For the given time interval, the method finds whether there is any relationship between numbers in these two strings. If there is no relationship, then all the NS values are recalculated by offsetting the ET value used for calculating NS by 1 unit. Then the process of finding a relationship is repeated. As the set of NS value patterns is extremely large, at some point the relationship between these two strings is found. This process is called synchronization.

Extending NSS into Future, i.e., Beyond Historical Data for Forecasting.

Synchronized NSS can be extended into the future. As a direct relationship between NS values and event values is established, the same can be used for predicting the event. This is possible because NS values can be extended infinitely into the future.

Building NSS for Each One of the Parameters.

The above process is used for forecasting values of each one of the parameters that have a bearing on the occurrence of the event. It means that one NSS is built for each one of the parameters.

Discrepancy.

When one or more of the parameter values become asynchronous with rest of the values, the event intensity will not be the same as predicted. This is called a discrepancy. The method builds a NSS for each one of the parameters for this reason only. This method will search all the forecasted parametric values for asynchronous ones. Based on those values, it will correct the event intensity forecast.

By intentionally changing one or more of forecasted parametric values, an event can be stopped from occurring or its intensity can be dramatically decreased/increased. This is the principal benefit that can be accrued from this method. This process is called optimization and is discussed below.

Optimization.

The optimizer module of the method assists the users in improving upon the forecasted results. The optimizer contains a built in simulator. This simulator provides user with an opportunity to perform “what if” analysis. When historical data are processed patterns between values of various parameters are collected. These patterns are analyzed by the optimizer module. During this process, the optimizer module frames rules for verifying validity of data of each parameter, in isolation as well as in combination with values of other parameters. Rules for verifying boundary values for each one of the parameters are also part of this rule set. The verifier is the sub-module that holds all these rules. The optimizer module provides the user with a facility to change values of various parameters (theoretically). Whenever the user makes such changes in the parametric value set, the verifier module validates these changes and throws back those changes inconsistent with historical data. At this stage the parameters whose values are changed become asynchronous with rest of the data. This causes a discrepancy in the forecasted event, i.e. the simulator shows that forecasted event does not occur at the predicted intensity at the predicted time and thus helps users in improving upon the forecasted results.

The optimizer also works in a fully automated mode. In this mode it provides the user with facility to enter a desired range both in intensity and period of occurrence of the event. Once these values are entered, it reconstructs the range of each one of the parametric values for the given time intervals. Now if values of all these critical parameter are kept within the stipulated range then it may become possible for the user to realize a desired event at a desired time.

The invention differs from prior art systems in that these Numeric Sequences are initially formulated without regard to the occurrence of the events at issue. They are instead raw patterns and numerous combinations of patterns relating to elapsed time. Only after these patterns and combinations are established in raw form is there any attempt to time them to the occurrence of the events at issue.

This historic data that is collected is appropriate for the predicted event that is the subject of the system. For example, in the case of disease outbreak, the past data would likely include the actual occurrences of the disease outbreak, along with data pertaining to causative or correlative factors such as weather, the prevalence and characteristics of carriers, and lifestyle and hygiene factors.

These variables are quantified so that the input is numerical. Disease can be quantified as diagnosed cases per population number, such as cases per thousand individuals, or in other desired quantifications such as deaths per population number. The method chosen for quantifying the input data should correspond to the desired prediction; if the prediction that is desired is deaths per 1000 population, then the input data should similarly be expressed in deaths per 1000 population.

Other factors are consistently quantified in like manner. Weather can be expressed in temperature, humidity and rainfall per chosen period. Variables that are not ordinarily expressed in number can be quantified arbitrarily; for example, seasons of the year can be expressed numerically as 1, 2, 3 or 4. Gender can be expressed as 1 or 2, and occupations of individuals can be assigned numeric codes. Each variable that appears to cause or correlate with the predicted event is preferably quantified and input as part of the historic data 12.

Each item of historic data 12 is matched with the time at which it occurred. The time scale begins with the earliest historic data at “0” and proceeds to the most recent available historic data. The time increment is chosen as appropriate for the data. If the data has a time precision that is no more than weekly, for example, the time increment could be one week. If the most precise data is expressed in terms of a precision of seconds or tenths of a second, then similar precision is appropriate for the time increment. Of course, this can produce relatively large numbers for the time scale; a time scale expressed in seconds will include numbers that are equal to the number of seconds in a year for the scale at the point of one-year elapsed. The computational issues presented in manipulating these large numbers are easily handled by modem numeric processors.

If location is a parameter of interest, data can be collected and input with the aid of geographical positioning systems (“GPS”) or global information systems (“GIS”). Data associated with the geographic data can be entered on-site using traditional methods, and the position associated with such data is entered automatically or by the simple press of a button which determines geographic position using GPS equipment and enters such position in the database. Similarly, GIS technology can be used which inherently associates location with other variables.

The data reader 14 facilitates the input of data from popular databases such as ORACLE, SYSBASE or INGRES brand. It is desired that the input historic data be set forth in a FOXPRO brand or flat data file for use by the system. The data reader can be equipped with web-enabled software of the kind known in the field to access data from remote servers via a network such as the Internet or a private network. The software may include a graphical user interface that allows the user to specify the fields for which the production models are required.

The diary of events module 16 works with the iterative generator to produce an n-dimension numeric space (“NDNS”) and a set of numeric sequence strands (“NSS”) within that space. The variables are “normalized,” meaning that they are graded on a finite scale such as 1-100 (including decimal fractions).

The system then identifies times in the past when the historic data shows the occurrence of the event that is the subject of the prediction process. If the predicted event is the outbreak of disease of a particular kind and magnitude, for example, the system identifies those instances in the past when that occurred. Each such instance can then be identified by its time coordinate T, wherein T=0 is the start of the historic data and the time scale runs forward from then. Such instances are notated ET₁, ET₂, ET₃ and so on for illustrative purposes. Time T is preferably expressed in the finest increment in which the historic data itself is expressed.

The numeric values of the many variables at several but less than all the instances at which the predicted event occurred in the past are then ascertained, and these values are used to calculate the numeric sequences NS using formulae. For example, if the predicted event occurred five times in the historic data, then numeric sequences NS could be calculated for two or three or four of such instances.

The goal is to calculate the many numeric sequence values such that they fall within a small range or band at ET₁, ET₂, ET₃, etc. This is done by initially calculating them with the earliest time T equal to zero. If the numeric sequences calculated using the data corresponding to these times fall within the chosen range or band, then one can proceed to the next step.

If the numeric sequences calculated using the earliest time set at zero do not fall within the band or range selected, then the initial time is offset by one time increment. If the time increment for the collected data is one week and the calculations are performed in seconds, then the initial time can be offset by the number of seconds in a week, i.e. 7×24×60×60. The process of calculating the numeric sequence values for the chosen instances at which the predicted event occurred in the past, using the new values of the variable time, is then repeated. If those numeric sequence values then fell within the selected band or range, the process goes to the next step. If not, the time variable is offset yet again and the calculations are repeated. This step is done repeatedly until the calculated numeric sequence values fall within the selected band or range. Stated another way, event E is defined as occurred if value of X crosses 100. Parameters PA and PB have bearing on the event E. Event E has occurred at times T1, T2, T3 and T4. Now the process begins as follows:

a. Time intervals ET1=T2−T1 and ET2=T3-T2 are taken into consideration.

b. Numeric sequence (NS 1) is started with time (t)=0.

c. Three values reflecting E are identified on NS 1, their time intervals are measured and if they match ET1, ET2, continue further down else go back to ‘b’ and restart by incrementing time.

d. By adding time interval T4-T3, NS 1 value will be checked. If it matches with E value at T4, then it means the sequence is suitable one and it is continued. Else go back to ‘b’ with further incrementing time.

e. Continue this process if further E values are available in past data, else simply predict future events (Where NS 1 values match E values, Time intervals help in predicting the time). The same process is applied for other parameters also.

This method has been successful in predicting the breakdown of a centrifuge used in chemical/pharmaceutical industries, batch failures in the bulk drug industry, the quality of manufactured bottles in the glass industry, batch failures in the paper industry due to various quality problems and the growth of virus on different cultures under laboratory conditions.

The next step is to calculate the numeric sequence values for the complete period for which data are available. The numeric sequence values are calculated for each incremental time including each time at which the predicted event occurred. If the numeric sequence values calculated for each time at which the predicted event occurred fall within the selected band or range, then the process proceeds to the next step. If not, then the process goes back to the step of once again offsetting the time value by one increment, and re-computing the numeric sequence values again. It should be recognized that each iteration refines the accuracy and efficiency of the system. These additional iterations take place with respect to past data collected at the outset, and also with respect to data that is subsequently collected as events occur.

Once the calculated numeric sequence values fall within the selected range or band for all predicted events, the process moves to the next step. The next step is to predict the predicted event in the future based on the occurrence and value of the variables in the numeric sequence.

Discrepancies may occur in the operation of the process, which are addressed as follows. Occasionally, there is a large discrepancy between the predicted event and the occurrence of the actual event in the historic data. For example, there may be substantial difference between the number of actual cases of a disease per population group and the number of predicted cases per population group. In that event, the process looks for a substantial aberration in the value of one of the factors in the input data. It may be, for example, that the amount of rainfall in the historic data corresponding to the discrepant prediction was extremely high or extremely low. However, the system can overcome this problem by building n-dimensional numeric sequences and synchronizing them for each one of the parameters that has a bearing on the occurrence of the event.

### EXAMPLE 1

This actual example utilizes the present invention to predict successfully the outbreak of Japanese Encephalitis (“JE”) in India. By changing the input parameters, the system can be used to predict the outbreak of AIDS, tuberculosis or other identified disease.

A principal vector of JE is known to be the mosquito Culex tritaeniorhyncus. Other vectors include Cx. vishnui group, Cx. pseudovishnui, Cx. bitaeniorhyncus, Cx. gelidus, Anopheles subpictus, An. hyrcanus, An. barbirostris and Mansonia annulifera. The incubation period for JE is 9-12 days in mosquitoes and is 5-15 days in man.

JE was reported in 1952 in India and was diagnosed in 1955 in the North Arcot district of Tamil Nadu and Chottor district of Andhra Pradesh. In 1978, it was isolated in CMC, Vellore. Occasional cases of JE have been earlier reported from adjoining areas of the South Arcot district as well as from Pondicherry. Between September and November 1981, an extensive JE epidemic was reported in the South Arcot district of Tamil Nadu and the Union Territory of Pondicherry. A total of 633 patients of whom 151 (23.8%) died were reported through the end of that November. The disease has been reported in many places in South India, the maximum incidence being 7,463 cases with 2,755 deaths (36.9%) in 1978. The worst affected states in India are Andhra Pradesh, Tamil Nadu and Karnataka from South India; UP, Bihar and West Bengal from North India; and Assam and Manipur from the Northeast region. In Andhra Pradesh, the disease was recorded almost every year in certain districts of Kurnool, Ananthapur, Guntur, Krishna, Prakasham, Nalgonda, Warangal, Cuddopah and Chittor. From 1990-99, a total of 5,609 cases with 2,256 deaths were reported in that state with an average case fatality rate of 40.22%. There is thus considerable historical data for the outbreak of this relatively common and serious disease.

The system of the present invention was used to build an n-dimensional numeric space based on the actual data. Time T was taken on the x-axis. The values of each one of the effective parameters was taken on the y-axis. The number of cases was marked on the z-axis. For each parameter there was one such space. For n parameters there were n ‘y’ axes. This is sometimes called n-dimensional space herein. This cannot be visualized and as such one can consider this as virtual space. A strand (not a straight line) connects all the events (in this case number of cases). A strand extender projects the existing strand into the future to forecast the number of cases that may occur in future.

Genetic algorithms are used for writing the software that creates and extends these NSS strands. This software is written using Java programming language. As such, it is platform free software and can be used on most of the machines.

Three years data (years 1997, 1998, 1999) pertaining to Kurnool District of Andhra Pradesh have been given as historic data input to the system. Average rainfall, Humidity, Maximum/Minimum temperatures, Crop practices, Irrigation facilities, Vector Density, Month, Year, etc., are among the information fed to the Engine. The modules used in this Example were data reader, diary of events, iterative generator, forecaster and communicator.

The data reader module facilitates inputting data from any one of the popular databases including ORACLE, SYSBASE, INGRES to FOXPRO or a flat data file. Web-enabled software such as Data Reader can access data from remote servers also. The Graphical User Interface (GUI) of this software enables the user to specify the fields for which the prediction models are required.

The diary of event module establishes relationship between the causative factors and the disease by reading data from data reader. The pattern recognition tool set of the software will establish relationship between various parameters and events occurred.

The iterative module works in tandem with diary of events module. It is based on data (the longer the data set period, the more accurate is the prediction) and generates both the n-dimensional numeric space (NDNS) and the corresponding numeric sequence strands (NSS). An iterative module, it generates and regenerates these NDNS/NSS combination until obtaining a satisfactory result. It uses Genetic Algorithms for generating NDNS as well as NSS.

Based on the data (the longer the data set period, the more accurate is the prediction), the iterative module generates the required logic into a software tool called Forecaster. This generates predictions on the occurrence of the future events.

Predictions are of both long term and short term in nature. The self-learning algorithms contained by the iterative module continuously improve the precision and accuracy of the predictions generated by it. In short—and this is important—the system is self-learning; the more it is used, the more accurate it becomes.

The variables used in this model are the following:

1. Number of Cases

2. Mosquito Abundance

3. Dusk Index

4. Infected Vector Abundance

5. Rainfall

6. Humidity

7. Maximum Temperature

8. Minimum Temperature

9. Wing Length of Mosquito

10. Wing Beat Frequency

11. Local Vegetation

12. Type of Residence

13. Water Resources

14. Habitat

15. Breeding Area

16. Age

17. Gender

18. Profession

19. Education

20. Awareness

21. Income Range

The numeric sequence NSI is used in this model. NSI is:

C values 4,9 table.

| | | | |
|---|---|---|---|
| 358.47583, | 35999.04975, | −0.00015, | −0.0000033, |
| 319.51913, | 19139.85475, | 0.000181, | 0.0, |
| 102.27938, | 149472.51529, | 0.000007, | 0.0, |
| 225.32833, | 3034.69202, | −0.000722, | 0.0, |
| 212.60322, | 58517.80387, | 0.001286, | 0.0, |
| 175.46622, | 1221.55147, | −0.000502, | 0.0, |
| 72.65000, | 428.380000, | 0.0, | 0.0, |
| 37.73000, | 218.460000, | 0.0, | 0.0, |
| 229.95000, | 144.913000, | 0.0, | 0.0/ |

D values 4,9 table

| | | | |
|---|---|---|---|
| 279.696680, | 36000.76892, | 0.0003025, | 0.0, |
| 293.737334, | 19141.69551, | 0.0003107, | 0.0, |
| 178.179078, | 149474.07078, | 0.0003011, | 0.0, |
| 238.049257, | 3036.301986, | 0.0003347, | −0.00000165, |
| 342.767053, | 58519.21191, | 0.0003097, | 0.0, |
| 266.564377, | 1223.509884, | 0.0003245, | −0.0000058, |
| 243.49747, | 429.863546, | 0.0003160, | −0.0000006, |
| 84.957994, | 219.885914, | 0.0003205, | 0.0000006, |
| 92.423, | 146.311, | 0.0, | 0.0/ |

E values 4,9 table

| | | | |
|---|---|---|---|
| 0.01675104, | −418.000, | −1.26, | 0.0, |
| 0.09331289, | 920.640, | −0.77, | 0.0, |
| 0.20561421, | 204.630, | −0.30, | 0.0, |
| 0.04833475, | 1641.800, | −4.676, | −0.017, |
| 0.00682069, | −477.400, | 0.91, | 0.0, |
| 0.05589232, | −3455.000, | −7.28, | 0.0074, |
| 0.04634440, | −265.800, | 0.77, | 0.0, |
| 0.00899704, | 63.300, | −0.02, | 0.0, |
| 0.24864400, | 0.000, | 0.00, | 0.0/ |

AS values 4,9 table

| | | | |
|---|---|---|---|
| 0.0, | 0.0, | 0.0, | 0.0, |
| 48.786442, | 0.7709917, | −0.0000014, | −0.00000533, |
| 47.145944, | 1.1852083, | 0.0001739, | 0.0, |
| 99.443414, | 1.01053, | 0.00035222, | −0.00000851, |
| 75.779647, | 0.8998499, | 0.00041, | 0.0, |
| 112.790414, | 0.8731951, | −0.00015218, | −0.00000531, |
| 73.477111, | 0.4986678, | 0.0013117, | 0.0, |
| 130.681389, | 1.098935, | 0.00024987, | −0.000004718, |
| 108.900000, | 1.3576, | 0.0, | 0.0/ |

ORR values 9 table

| | | |
|---|---|---|
| 1.000000, | 1.5236883, | 0.3870986, |
| 5.202561, | 0.7233316, | 9.5547460, |
| 19.218140, | 30.1095700, | 39.5177380/ |

INC values 9 table

| | | | |
|---|---|---|---|
| 0.000, | 1.850, | 7.003, | 1.309, |
| 3.394, | | | |
| 2.493, | 0.772, | 1.779, | 17.150/ |

EPS=23.452294−0.0130125*T

REPEAT THE FOLLOWING 9 TIMES CHANGING I VALUE

FROM 1 T0 9

AN(I)=(C(1,I)+C(2,I)*T+C(3,I)*T*T+C(4,I)*T*T*T)
END OF THE REPEAT
X=3*AN(4)−8*AN(2)+4*AN(1)
X=0.0113*SIND(X)+0.009*COSD(X)
Y=AN(4)−AN(2)

C1=(7*COSD(Y−49)+6*COSD(AN(4)+Y−188)+4*
COSD(2*(Y−96)))/1E3

V=T/5+0.1
P=237.47555+3034.9061*T
Q=265.91650+1222.1139*T
Z=Q−P
VV=5*Q−P−P
SV=SIND(VV)
S2V=SIND(VV+VV)
CV=COSD(VV)
SZ=SIND(Z)
S2Z=SIND(Z+Z)
S3Z=SIND(Z+Z+Z)
CZ=COSD(Z)
C2Z=COSD(Z+Z)
C3Z=COSD(Z+Z+Z)
SQ=SIND(Q)
S2Q=SIND(Q+Q)
CQ=COSD(Q)
C2Q=COSD(Q+Q)

AA=(331*SV−64*V*CV+14*SZ+18*S2Z+7*S3Z+
(7*SZ+6*S2Z−34*Cz)*SQ++ (−36*SZ−6*CZ−7*C2Z)*
CQ)/1E3
BB=(7*SV−20*CV+(7*SZ−4+34*CZ+6*C2Z)*SQ+
(38*SZ+6*S2Z−7*CZ)*CQ−5*SZ+ *S2Q+6*CZ*C2Q)/1E3
CC=(36*SV+13*CV+(−68*SZ−11*S2Z−2+13*CZ)*
SQ+(15*SZ−8+60*CZ+10*C2Z++5*C3Z)*CQ+
(−10*SZ−10*CZ)*S2Q+(−10*SZ+5*S2Z+10*CZ)*C2Q)/1E5
SA=(−814*SV+18*V*SV−10*CV+161*V*CV+8*SV−149*
SZ−40*S2Z−15*S3Z−6*SQ++(9*SZ−17*S2Z−6*S3Z+
81*CZ+15*C2Z)*SQ+(86*SZ+25*CZ+14*C2Z+6*C3Z)+
*CQ+(6*SZ+9*S2Z)*S2Q+(−5*CZ−8*C2Z)*C2Q)/1E3
SB=(77*SV+45*CV−15*V*CV−7*SZ+(−76*SZ−25*S2Z−9*S3Z)*
SQ+(−73−150*CZ++27*C2Z+10*C3Z)*CQ+(−14*SZ−8*CZ+
13*C2Z)*S2Q+(−14*SZ+12*S2Z+15+ *CZ−3*C2Z)*C2Q)/1E3
SC=(−79*SV+26*V*SV+134*CV+12*V*CV+(124+266*CZ−47*C2Z−
19*C3Z)*SQ++(−127*SZ−42*S2Z−15*S3Z−13*V*CZ)*CQ+(22*SZ−22*
S2Z−6*S3Z−28*CZ++20*C2Z)*S2Q+(−28*SZ−16*CZ+22*C2Z+
6*C3Z)*C2Q)/1E5
DD=(57*SV+293*CV+3363*CZ−308*C2Z−142*C3Z+
(110−281*SZ+69*S2Z−39*S3Z++214*CZ−100*C2Z−64*C3Z)*SQ+
(−89+221*SZ−159*S2Z−65*S3Z+289*CZ++217*C2Z)*CQ+
(−78*CZ+50*C2Z)*S2Q−86*SZ*C2Q)/1E5

REPEAT THE FOLLOWING CHANGING J VALUE
FROM 1 TO 9
ORRR=ORR(J)
ANN=AN(J)
ECC=E(1,J)+(E(2,J)*T+E(3,J)*T*T+E(4,J)*T*T*T)/1E7
INCL=INC(J)
ASN=AS(1,J)+AS(2,J)*T+AS(3,J)*T*T+AS(4,J)*T*T*T
MNLN=(D(1,J)+D(2,J)*T+D(3,J)*T*T+D(4,J)*T*T*T)
IF(J.EQ.2) ANN=ANN−X
IF(J.EQ.2) MNLN=MNLN−X
IF(J.EQ.4) MNLN=MNLN+AA
IF(J.EQ.4) ECC=ECC+CC
IF(J.EQ.4) ANN=ANN+AA−BB/ECC
IF(J.EQ.6) MNLN=MNLN+SA
IF(J.EQ.6) ECC=ECC+SC
IF(J.EQ.6) ANN=ANN+SA−SB/ECC
IF(J.EQ.6) ORRR=ORRR+DD
IF(J.EQ.7) MNLN=MNLN−0.816−0.166*SIND(MNLN+50.)
IF(J.EQ.8)

MNLN=MNLN+0.600−0.100*SIND(MNLN/2−90.)+.166*(T−1.)

IF(J.EQ.9) MNLN=MNLN−0.100*SIND(MNLN)
EC=ECC/0.0174532925
EOLD=ANN
ITR=0

ANEC=EOLD+(ANN+EC*SIND(EOLD)−EOLD)/
(1.−ECC*COSD(EOLD))

ITR=ITR+1
ADIF=(EOLD−ANEC)*100./EOLD
EOLD=ANEC
ANTR=

(ATAND(SQRT((1+ECC)/(1−ECC))*TAND(ANEC/2.))*2.)

U=MNLN+ANTR−ANN−ASN
UU=ATAND(COSD(INCL)*TAND(U))
TRLONG=UU+ASN
TRLONG=TRLONG
RAD=ORRR*(1.−ECC*COSD(ANEC))
ERAD=RAD
ELONG=TRLONG
ANG(1)=TRLONG
BETA=ASIND(RAD/ELONG*SINB)

DECL(J+1)=ASIND(SIND(BETA)*COSD(EPS)+COSD(BETA)*
SIND(EPS)*SIND

+ (ANG(J)))
END OF REPEAT
SUM=0
REPEAT THE FOLLOWING CHANGING I VALUE FROM 1 TO 9
SUM=SUM+DECL(I)
END OF REPEAT
NS(1)=SUM/9

NS2:

A values 1 to 14

26.,22.,18.,14.,10.,6.,2.,10.,6.,2.,26.,22.,18.,14./

B=0.276 919 398+100.002 1359*T+0.000 001 075*T*T
+( 1.002 737 908*15.)/24.
E=23.452294−0.0130125*T
Z=ATAN2D(TAND(B),COSD(E))
X=1.002 737 908
D=219180.*T+26543.

REPEAT THE FOLLOWING VARYING I FROM 1 TO 14

F=D*SIND(A(I))−2*B*SIND(A(I))+4*E*Z*SIND(A(I))*COSD(A(I))

NS(2)=NS(2)+F/X+4
END OF REPEAT
NS(2)=NS(2)/14

NS3:
A=358.47583+35999.04975*T−0.00015*T*T−0.0000033*T*T*T

B=279.69668+36000.76892*T+0.0003025*T*T
C=0.01675104−0.0000418*T−0.000000126*T*T
T=2*B
S=B+(1.91946−0.004789*T−0.000014*T*T)*SIND(A)

+(0.020094−0.0001*T)*SIND(A+A)+0.000293*SIND(3.*A)

E=(23.452294−0.0130125*T)
D=ASIND(SIND(E)*SIND(S))
Y=TAND(E/2.)**2
NS3=Y*SIND(T)−2*C*SIND(A)+4*C*Y*SIND(A)*COSD(T)
NS3=(E−0.5*Y*Y*SIND(T+T)−1.25*C*C*SIND(A+A))
NS3=NS3/0.0174532925/15.

NS4:

O=(259.183275−1934.142*T+0.002078*T*T)
L=(270.434164+481267.883*T−0.001*T*T
+0.004*SIND(346.56+T*132.87))
M=(296.104608+477198.8491*T*O)
D=(350.737486+445267.1142*T)
F=(11.250889+483202.0251*T)
MM=(358.47583+

35999.04975*T−0.00015*T*T−0.0000033*T*T*T)

E=1−0.002495*T−0.00000752*T*T
M2=MM+MM
D2=D+D
F2=F+F
A=L+(6289*SIND(MM)+1274*SIND(D2−MM)+658*SIND(D2)

+214*SIND(M2)−114*SIND(F2)+59*SIND(D2−M2)+53*
SIND(D2+MM)−35*SIND(D)+15*SIND(D2−F2)−13*SIND(F2+MM)−
11*SIND(F2−MM)+11*SIND(4*D−MM)+10*SIND(3*MM)+9*
SIND(4*D−M2)+E*(−186*SIND(M)+57*SIND(D2−M−MM)+46*
SIND(D2−M)+41*SIND(MM−M)−30*SIND(MM+M)−8*
SIND(M−MM+D2)−7*SIND(D2+M)+5*SIND(M+D)+4*
SIND(MM−M+D2)+3*SIND(M2−M)+2*(SIND(D2−M−M2)−
SIND(M2+M))+SIND(4*D−M−MM)))
B=(5128*SIND(F)+281*SIND(MM+F)+278*SIND(MM−F)+173*
SIND(D2+F)+55*SIND(D2+F−MM)+46*SIND(D2−F−MM)+
33*SIND(D2+F)+17*SIND(M2+F)+9*SIND(D2+MM−F))
NS4=ASIND(SIND(B)*COSD(E)+COSD(B)*SIND(E)*SIND(A))

The system generates heuristic for accurately assessing the geographical location of the outbreak of any vector-borne diseases. It also demarcates the endemic area (sq.km) where the people are prone to the infection, in the specific outbreak. This is very important; not only does the system predict disease outbreaks, but it predicts with some precision the locations of the outbreak.

The communication module of the system also is capable of informing all the concerned authorities and agencies about the impending outbreak and its magnitude. The communication module requires a good PSTN; if Internet facility is available it will use the facility.

This is highly scaleable software and as such can handle any volume of data. It can be integrated with any existing software across a wide range of hardware/operating platforms. The system is thus extremely robust.

The forecasts of the year 1997, 1998 and 1999 are given in tables 1, 2 and 3 respectively.

TABLE 1
J.E. incidence in Kurnool district
actual figures and HE forecast for the year 1997

| | Number of cases | Number of cases | % Accuracy of |
|---|---|---|---|
| Month | actually recorded | predicted by HE | HE predictions |
| Jan. | Nil | Nil | 100 |
| Feb. | Nil | Nil | 100 |
| Mar. | Nil | Nil | 100 |
| Apr. | Nil | Nil | 100 |
| May | Nil | Nil | 100 |
| Jun. | Nil | Nil | 100 |
| Jul. | Nil | Nil | 100 |
| Aug. | Nil | Nil | 100 |
| Sept. | 6 | 7 | 85.71 |
| Oct. | 46 | 61 | 75.41 |
| Nov. | 206 | 291 | 70.79 |
| Dec. | 122 | 141 | 86.52 |

TABLE 2
J.E. incidence in Kurnool district
actual figures and HE forecast for the year 1998

| | Number of cases | Number of cases | % Accuracy of |
|---|---|---|---|
| Month | actually recorded | predicted by HE | HE predictions |
| Jan. | Nil | Nil | 100 |
| Feb. | Nil | Nil | 100 |
| Mar. | Nil | Nil | 100 |
| Apr. | Nil | Nil | 100 |
| May | Nil | Nil | 100 |
| Jun. | Nil | Nil | 100 |
| Jul. | 7 | 9 | 77.78 |
| Aug. | 4 | 8 | 50.00 |
| Sept. | 18 | 27 | 66.67 |
| Oct. | 50 | 61 | 81.97 |
| Nov. | 29 | 34 | 85.29 |
| Dec. | Nil | Nil | 100 |

TABLE 3
J.E. incidence in Kurnool district
actual figures and HE forecast for the year 1999

| | Number of cases | Number of cases | % Accuracy of |
|---|---|---|---|
| Month | actually recorded | predicted by HE | HE predictions |
| Jan. | Nil | Nil | 100 |
| Feb. | Nil | Nil | 100 |
| Mar. | Nil | Nil | 100 |
| Apr. | Nil | Nil | 100 |
| May | Nil | Nil | 100 |
| Jun. | Nil | Nil | 100 |
| Jul. | Nil | Nil | 100 |
| Aug. | 1 | 2 | 50.00 |
| Sept. | 2 | 3 | 66.67 |
| Oct. | 75 | 97 | 77.32 |
| Nov. | 166 | 192 | 86.46 |
| Dec. | 25 | 32 | 78.13 |

The predictions of the years 2000, 2001 and 2002 are given in table 4. The phasewise forecastings for the years 2000, 2001, and 2002 are given in table 5.

TABLE 4
Forecast of J.E. incidence in Kurnool district
for the years 2000, 2001 and 2002

| | Forecast regarding | Forecast regarding | Forecast regarding |
|---|---|---|---|
| | number of cases | number of cases | number of cases |
| Month | in the year 2000 | in the year 2001 | in the year 2002 |
| Jan. | Nil | Nil | Nil |
| Feb. | Nil | Nil | Nil |
| Mar. | Nil | Nil | Nil |
| Apr. | Nil | Nil | Nil |
| May | Nil | Nil | Nil |
| Jun. | Nil | Nil | Nil |
| Jul. | 9 | Nil | 4 |
| Aug. | 9 | 7 | 6 |
| Sept. | 22 | 12 | 9 |
| Oct. | 71 | 69 | 54 |
| Nov. | 37 | 312 | 32 |
| Dec. | 25 | 147 | 14 |

TABLE 5
Phasewise Forecast of J.E. in Kurnool District,
Andhra Pradesh for the years 2000, 2001 and 2002

| Parameters | Year 2000 | Year 2001 | Year 2002 |
|---|---|---|---|
| Peak abundance of | Sept. 24, 2000 | Sept. 27, 2001 | Sept. 25, 2002 |
| Culex sps (Phase I) | to | to | to |
| | Oct. 10, 2000 | Oct. 10, 2001 | Oct. 10, 2002 |
| Multiplication of | Oct. 11, 2000 | Oct. 11, 2001 | Oct. 11, 2002 |
| JE virus in reservoirs | to | to | to |
| (Phase II) | Oct. 23, 2000 | Oct. 23, 2001 | Oct. 23, 2002 |
| Intrinsic incubation | Oct 24, 2000 | 19-24-2001 | Oct. 24, 2002 |
| period in human | to | to | to |
| beings | Nov. 5, 2000 | Nov. 5, 2001 | Nov. 5, 2002 |
| (Vaccination Phase III) | | | |

The system forecasts the following:

1. The period when the vector is abundant. If vector control measures are taken during this period, the outbreak of the disease can be minimized. In fact, it can be reduced to almost negligible levels.

2. The period when these mosquitoes get infected through biting reservoirs such as pigs, donkeys, cattle, etc. Extrinsic incubation occurs in mosquitoes during this period.

3. The period when these mosquitoes bite human beings. Intrinsic incubation in human beings occurs in this period. (vaccination period)

4. The number of positive cases of JE in the district (if preventive measures are not taken during the period mentioned in the first point).

However the number of possible cases predicted by the system can be reduced to almost zero by taking preventive measures, especially during the period of peak vector density. As this specific period lasts only for about two to three weeks, health agencies can take extensive vector control measures. This action will result in reducing the number of cases drastically, thus saving a number of lives.

Necessary vector control measures to be taken to reduce the number of JE cases significantly will be at Phase I. This allows widening the gap between the man vector contract and the transmission.

Necessary measures must be taken to avoid the presence of reservoirs like pigs, donkeys, etc., in the environment, so that the multiplication of JE virus can be reduced which will bring down the rate of transmission of JE virus to the human beings.

Although the Phase III, i.e., intrinsic incubation period in human beings is too late to control JE, proper vaccination will help in reducing the number of deaths out of positive cases in the particular period.

Other Uses.

As mentioned above, numerous other applications are available for the method and system of the present invention within the field of predicting disease outbreak and in many other fields. In each application, the predicted event or occurrences associated with the event is a parameter. Plotted in two dimensional space are Numeric Sequences and Elapsed Time, and these two dimensional plots are overlaid in multi-dimensional x-y axes with an integrated paradigm. In agriculture, the system is effective in forecasting the occurrence of crop and livestock blight and disease. Armed with relatively accurate forecasts, farmers can take preventive measures such as applying pesticides or altering planting techniques or timing, or changing crops. Moreover, such forecasts can be used to increase the production of pesticides, to store alternative food supplies or to hedge commodities.

In the pharmaceutical industry, vast amounts of research and money is expended in devising and testing new drugs. Many of the properties of these drugs are a function of the three dimensional shape of the molecules comprising them. Some of these molecules, such as amino acids, are highly complex. Software is already available to simulate the chemical and physical binding of molecules for the purpose of viewing probable shapes, but the true properties of an engineered drug currently can be ascertained only by producing and testing real drugs. This is extraordinarily expensive, and the test results are frequently disappointing; as much as 80% of new drugs fail in trials. The present system, however, can produce surprisingly accurate predictions based on past data.

### EXAMPLE

Peptide drugs lack activity orally because they are digested, and they often lack selectivity because they react with many receptors. Therefore it is necessary to transform active peptide compounds into active non-peptide drug compounds, which can be a difficult task. Here the invention helps find active compounds by using relationships between chemical structures and their biological activities. By establishing patterns between chemical structures and their biological activities, the invention can speed the iterative process of drug discovery, in which new compounds bring new information.

First, one generates relationship patterns from structure and activity data, then searches past data for compounds that match. Once this is done then one can design new molecules to fit a hypothesis and synthesize and assay the most promising candidates.

Again, in the absence of a receptor structure, from which one can construct new compounds, the invention can be used for developing new design techniques that must infer a cavity from available active leads. A useful approach is to build a receptor-surface model (a model for the receptor site) and to construct compounds inside this model that fit sterically and complement the putative receptor interactions. The forecaster model of the invention can predict the possible model that can fit thus hastening new molecule development process. Invention in conjunction with available traditional software can be a powerful tool for new drug design. It can be used to fit molecules into the active site of a receptor by identifying and matching complementary polar and hydrophobic groups. As empirical functional software the invention can be used to prioritize the hits.

In the field of Customer Relationship Management (sometimes referred to as “CRM”), the system has wide applicability. By tracking purchasing patterns for individual customers and groups of customers, and generating suitable NSS indicators, the system can predict with surprising accuracy a given customer's purchases or interests over a future time period. This allows vendors to present to a customer the particular types of goods and services that the customer is interested in purchasing, at the particular time that the interest is ripe. in electronic and other retailing, especially at e-commerce sites, large amounts of data is accessible regarding the interests and buying habits of individual and groups of customers. The amount of data, in fact, is so considerable that it exceeds the ability of existing techniques to process it effectively. The present system can apply a set of numeric sequence strands to such data to generate relatively reliable predictions of what an individual customer is likely to purchase during a given period of time in the future and the probable volume of his purchases. It will also indicate the price sensitivity of customers, the general types of goods and services the customer be interested in, and the cost/benefit analysis of focused marketing for individual customers. The system can also use historic data to optimize the formatting of an e-commerce site, by the positioning of captions and product service names on the screen, by appropriate color selections, and by formulating mailing lists.

Equipment failures are a costly problem in many manufacturing and other industries. The present invention addresses this by developing an archive of data in the form of history cards for pieces of equipment, containing service details and performance data, and then processing this data through appropriate numeric sequence standards. This can be used not only to evaluate and predict the performance of specific equipment studying alone, but also in relation to other interrelated equipment. For example, as any car owner knows, the performance and reliability of parts and equipment is often related to the performance and reliability of associated parts and equipment. The vibration produced by a failing motor can stress the motor mounts, and a poorly tightened screw can produce undue strain on the other screws in an assembly. A replacement part can result in unforeseen impacts on other parts, and even the replacement procedure itself can impact other elements. The present system can consider all these variables and parameters in predicting the need for service and maintenance.

In newer utility service, the operation of a power grid can be optimized by forecasting consumer demand, by predicting equipment failure, and by forecasting transmission and distribution losses. All these can be derived with considerable accuracy based on past data and appropriately tailored numeric sequence strands. For example, in forecasting demand, the method can predict hourly demand on a unit basis well in advance. This allows utility companies to optimize power procurement from feeder units. The lead time available through this method allows utilities to take necessary actions to eliminate load mismatches. The forecasting of equipment failures allows utilities to shift from time-based maintenance, i.e. maintenance conforming to a time schedule regardless of actual need, to event-driven maintenance, i.e. maintenance performed when actually needed. This can dramatically reduce maintenance costs by reducing unnecessary maintenance, at the same time as it dramatically reduces equipment failures by ensuring that maintenance is performed when necessary. In forecasting transmission and distribution losses, the users can predict future power losses and load mismatches well in advance, and assist them in identifying the most economical and effective solutions.

In conventional computer design, arithmetic operations are performed by compliment methods, each of which consumes a number of T-cycles. In contrast, the present invention can perform computations by pattern recognition. This greatly saves in processing power and time.

One of the most important commercial values of the invention can be accrued from its ability to increase the computing speeds of microprocessors. Many tasks such as searching the Internet, modeling the national economy, forecasting the weather—strain the capacities of even the fastest and most powerful computers. The difficulty is not so much that microprocessors are too slow; it is that computers are inherently inefficient. Modem computers operate according to programs that divide a task into elementary operations, which are then carried out serially, one operation at a time. Computer designers have tried for some time to coax two or more computers (or at least two or more microprocessors) to work on different aspects of a problem at the same time, but progress in such parallel computing has been slow and fitful. The reason, in large part, is that the logic built into microprocessors is inherently serial. (Ordinary computers sometimes appear to be doing many tasks at once, such as running both a word-processor and a spreadsheet program, but in reality the central processor is simply cycling rapidly from one task to the next.)

One way to solve this problem is to enable processors to do computing based on patterns. And the present invention does the same. One of the most important commercial values of this method can be accrued from its ability to increase the computing speeds of microprocessors. Number crunching is one of the principal activities of a microprocessor. It actually performs these arithmetic operations using one of the complementing methods. Performing these operations take large number of processing cycles of time measured in FLOPS (Floating-Point Operations per Second). With the help of this invention these microprocessors can find the exact pattern of the solution without actually performing the arithmetic. Thus this revolutionary technology can substantially increase the speed of microprocessors in a way that was never through of hitherto.

Take the example of division of 1 by 19. Instead of actually dividing, we can simply write a pattern of the solution like

1/19=0.052631578947368421.

In this example the pattern of the solution begins with 1 in the units place. Doubling current digit and adding carry digit obtain each next digit.

| | |
|---|---|
| The digit in units place is | = 1 |
| 1*2 (no carry digit) | = 2 |
| 2*2 = 4 (no carry digit) | = 4 |
| 4*2 = 8 (no carry digit) | = 8 |
| 8*2 = 6 (carry digit = 1) | 6 |
| 6*2 (12 + previous carry 1 = 13 and carry is 1) | = 3 |
| 3*2 = (6 + previous carry 1) | = 7 |
| . . . | |
| . . . | |

This process is continued until encountering recurrence of the same pattern. The method of this invention uses a very large number of patterns such as these. These patterns can be implemented on a microchip, most of the complicated numeric processing can be done with this add-on chip, thus substantially increasing the processing speed. The present invention can not only increase the speed of current processors but also the processors based on quantum computing technologies of future.

Theoretically, the prospects are good. Through the patterns of this method a processor can generate algorithms that could factor 140-digit-long numbers a substantially faster rate than is currently possible. Besides, this method can remarkably increase the performance of an internet search engine and help in shortening the time to unscramble encrypted transmissions.

A similarly subtle approach has been devised for factoring large numbers. Factoring is what computer scientists call a one-way problem: hard in one direction but easy in the other. Suppose the question is asked, “which two integers can be multiplied to obtain the number 40,301?” Systematically testing all the candidates might keep one busy for fifteen minutes or so. But if asked to multiply 191 by 211, it would take only about twenty seconds with pencil and paper to determine that the answer is 40,301. The lopsided difficulty of factoring compared with multiplication forms the basis for practical data encryption schemes such as the RSA protocol. Large prime numbers—say, a hundred digits each or so—make good “passwords” for such systems because they are easy to verify: just multiply them together and see whether their product matches a number that is already stored or that might even be made publicly available. Extracting the passwords from a 200-digit composite product of two large primes, however, is equivalent to factoring the large composite number—a problem that is very hard, indeed. The largest number that ordinary supercomputers have been able to factor with traditional algorithms is “only” 140 digits long.

However, when by using the patterns from this method and not actually performing the arithmetic in a traditional way the computer does, factoring can be done simply as efficient as multiplication. In computer science, one often tries to solve hard problems by converting them into simpler problems that one already knows how to solve. In similar way this problem can be converted into one of estimating the periodicity of a long sequence. Periodicity is the number of elements in the repeating unit of a sequence. The sequence 0, 3, 8, 5, 0, 3, 8, 5, . . . , for instance, has a periodicity of four. To estimate periodicity, a classical algorithm must observe at least as many elements as there are in the period. Whereas the pattern library of this method does much better. It identifies all the possible repeating sequence. A single pattern search operation then identifies the value of the sequence to which the answer corresponds. This is the beauty of the system.

Using this technology, the system can also optimize using the paradigm in the forecasting technologies. The optimization can be used throughout all industries, including but not limited to the pharmaceutical industry, in design, testing, synthesizing and manufacturing new therapeutic molecule's and compounds, in increasing computer processors, in optimizing power grid operations and consumption, in consumer conservation of energy, in optimization of manufacturing process as well as customer relationship management, and in inventory control. To this end the users can conduct operations more efficiently and effectively whether in marketing, manufacturing or sales of any products or services or in any other business that uses processes or that has customers.

This invention establishes through prediction modeling, relationships and interplays between datasets and creates and draws from the internal patterns of its software's library. It creates a pattern, so the user can identify the proposed outcome, which can be predetermined so that a change in the data or in the inputs can change the final or actual outcome.

1. A method of designing pharmaceutical treatment, comprising:
(a) identifying a set of formulae that are mathematical functions independent of pharmaceutical performance;
(b) establishing a pattern based upon said formulae;
(c) calculating a set of values based on the composition of a pharmaceutical;
(d) comparing said pattern to said set of values to establish a relationship; and
(e) extending said relationship to predict performance of the pharmaceutical.

2. The method of claim 1, further comprising identifying one or more variables contributing to said performance, and altering at least one identified variable to alter said predicted pharmaceutical performance.

3. The method of claim 1, further comprising identifying a desired pharmaceutical performance, and wherein said altering step includes altering said identified variable in order to encourage said desired performance by the pharmaceutical.

4. The method of claim 1, wherein said calculating step is based also, at least in part, on known properties of identified compounds or molecules of the pharmaceutical.

5. The method of claim 1, wherein said calculating step is based also, at least in part, on known interactivity of identified compounds or molecules of the pharmaceutical.

6. The method of claim 1, wherein said calculating step is based also, at least in part, on known surface configurations of identified compounds or molecules of the pharmaceutical.

7. The method of claim 1, further comprising using said method to predict the results of pharmaceutical treatment regimes.

8. The method of claim 1, further comprising using said method to optimize treatment regimes.

9. The method of claim 1, wherein said method is used to predict the performance of a pharmaceutical.

10. The method of claim 1, wherein said event is a parameter upon which one or more other events depend, and the method of claim 1 is performed for each such parameter.

11. A method of designing pharmaceutical treatment comprising:
(a) developing a set of formulae that are mathematical functions independent of pharmaceutical performance;
(b) Establishing a mathematical relationship between performance of a pharmaceutical and a combination of one or more of said formulae;
(c) extending said relationship to predict pharmaceutical performance;
(d) identifying one or more variables in said mathematical relationship that contribute to a predicted pharmaceutical performance; and
(e) altering at least one identified variable to alter said predicted pharmaceutical performance.

12. The method of claim 11, further comprising optimizing said method by repeated application.
